(19) United States
(12) Patent Application Publication
(10) Pub. No.: US 2026/0289752 A1
(43) Pub. Date: Sep. 24, 2026

(54) COMPUTED TOMOGRAPHY (CT) K-EDGE ENHANCED IMAGE RECONSTRUCTION OF A COMBINATION OF A PLURALITY OF WEIGHTED ENERGY BINS

(71) Applicant: GE Precision Healthcare LLC, Waukesha, WI (US)

(72) Inventors: Zhye Yin, Niskayuna, NY (US); Jiahua Fan, New Berlin, WI (US); Ming Yan, Hartland, WI (US)

(73) Assignee: GE Precision Healthcare LLC, Waukesha, WI (US)

(21) Appl. No.: 19/085,341

(22) Filed: Mar. 20, 2025

(51) Int. Cl. G06T 5/73; A61B 6/03; A61B 6/42; G06T 11/00

(52) U.S. Cl. CPC G06T 5/73; A61B 6/035; A61B 6/4241; G06T 12/00; G06T 2207/10081; G06T 2207/20192; G06T 2207/30004; G06T 2210/41; G06T 2211/40

(57) ABSTRACT

A computed tomography (CT) imaging system includes a polychromatic X-ray source, a photon counting detector configured to detect X-ray photons traversing a subject and one or more radiocontrast agents administered to the subject and bin the detected X-ray photons across a plurality of energy bins based on energies of the detected X-ray photons (where one or more radiocontrast agents include one or more K-edges in the diagnostic imaging energy range), memory including reconstruction bin weights configured to enhance one or more K-edges, suppress one or more K-edges, or enhance one or more K-edges and suppress one or more K-edges, and a reconstructor configured to apply the reconstruction bin weights to the plurality of energy bins, combine the weighted energy bins into a single set of data, and reconstruct the single set of data to generate K-edge enhanced volumetric image data that enhances at least one K-edge.

### FIELD

The following generally relates to computed tomography (CT), and more particularly to K-edge enhanced image reconstruction of a combination of a plurality of weighted energy bins.

### BACKGROUND

A non-spectral computed tomography (CT) scanner has included a single broadband X-ray tube mounted on a rotatable gantry opposite one or more rows of indirect conversion (e.g., scintillator/photodiode based) detectors. The X-ray tube rotates around a bore located between the X-ray tube and the one or more rows of detectors and emits polychromatic radiation that traverses the bore and a subject therein. With a peak tube voltage of 120 kVp (kilovolt peak), the energy spectrum of the emitted X-ray radiation (after filtering of low energy photons) may be from 40 to 120 keV (kiloelectron volt). The one or more rows of detectors detect radiation that traverses the examination region and generates projection data (line integrals) indicative thereof. The projection data is reconstructed to generate volumetric image data. Generally, corrections (e.g., scatter correction, beam hardening correction) are applied during reconstruction.

The voxels of the reconstructed volumetric image data are displayed as two-dimensional (2-D) images or three-dimensional (3-D) images using gray scale values corresponding to relative radiodensity. The gray scale values reflect the attenuation characteristics of the scanned subject and generally show structure such as anatomical structures within the scanned subject. Since the attenuation of an X-ray photon by a material is dependent on the energy of the X-ray photon traversing the material, the X-ray detected radiation also includes spectral information, which provides additional information indicative of the elemental and/or material composition of the scanned material of the subject. However, the projection data does not reflect the spectral characteristics as the values of the projection data are proportional to the energy fluence integrated over the energy spectrum, and the volumetric image data does not reflect the energy dependent information.

A spectral (multi-energy) CT scanner is further configured to detect the spectral information. With one example approach, the detector includes a direct conversion photon counting detector, which directly convert X-ray photons to electrical signals indicative of an energy of the X-ray photon, measure the energy of the individual X-ray photons from the signals, and group the individual X-ray photons into energy bins based on the measured X-ray photon energies. This allows for identifying K-edges (i.e., an abrupt increase in photoelectric absorption of X-ray photons at an energy level just beyond the binding energy of the K-shell electrons of a material) of certain radiocontrast agents, such as Iodine (I), Gadolinium (Gd), Tantalum (Ta), etc., and visually enhancing and/or suppressing a radiocontrast agent in the reconstructed volumetric image data.

For example, a K-edge image subtraction approach has been utilized to create images that visually enhance a radiocontrast agent. With one approach, an image is reconstructed with the projection data from an energy bin with an upper energy threshold at the K-edge energy of a radiocontrast agent of interest, another image is reconstructed with the projection data from a neighboring energy bin with a lower energy threshold at the K-edge energy, and the two images are subtracted to create a K-edge subtraction image. With relatively narrow energy windows for X-ray photons before and after the K-edge, the attenuation values of a material (other than the radiocontrast agent) in the two images are similar and subtract out, whereas the attenuation values of radiocontrast agent in the two images are different, due to the K-edge and relative to the other materials, and do not subtract out, and the subtraction has an overall effect of visually enhancing the radiocontrast agent in the K-edge subtraction image.

However, such a K-edge subtraction approach requires at least two reconstructions, one for each of the two images, including one for the higher energy bin and one for the lower energy bin. Unfortunately, the additional reconstruction consumes additional processing resources (e.g., memory, processing cycles, etc.) and delays generation of the final K-edge subtraction volumetric image data relative to a single reconstruction. Furthermore, each of the two reconstructions utilize counts from only a single energy bin, increasing noise and artifact relative to using all of the binned counts. Unfortunately, noise and artifact degrade image quality and can degrade image quality to a point where the image quality of the K-edge subtraction image is not well-suited (e.g., impractical, unworkable, etc.) for diagnostic imaging. In view of at least the foregoing, there is an unresolved need for another approach for K-edge imaging that mitigates at least the above-noted issues and/or shortcomings.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a computed tomography imaging system configured for at least spectral imaging includes a polychromatic X-ray source. The computed tomography imaging system further includes a photon counting detector configured to detect X-ray photons traversing a subject and one or more radiocontrast agents administered to the subject and bin the detected X-ray photons across a plurality of energy bins based on energies of the detected X-ray photons. The one or more radiocontrast agents include one or more K-edges in the diagnostic imaging energy range. The computed tomography imaging system further includes memory including reconstruction bin weights configured to enhance one or more K-edges, suppress one or more K-edges, or enhance one or more K-edges and suppress one or more K-edges. The computed tomography imaging system further includes a reconstructor configured to apply the reconstruction bin weights to the plurality of energy bins, combine the weighted energy bins into a single set of data, and reconstruct the single set of data to generate K-edge enhanced volumetric image data that enhances at least one K-edge.

In another aspect, a computer-implemented method includes binning X-ray photons, which are detected during a CT imaging acquisition utilizing a polychromatic X-ray source and a photon counting detector, across a plurality of energy bins based on energies of the detected X-ray photons. The computer-implemented method further includes applying a set of reconstruction bin weights to the plurality of energy bins, wherein the set of reconstruction bin weights are configured to enhance one or more K-edges, suppress one or more K-edges, or enhance one or more K-edges and suppress one or more K-edges. The computer-implemented method further includes combining the weighted energy bins into a single set of data. The computer-implemented method further includes reconstructing the single set of data to generate K-edge volumetric image data that enhances at least one K-edge.

In another aspect, a computer readable medium is encoded with computer executable instructions. The computer executable instructions, when executed by a processor, cause the processor to bin X-ray photons, which are detected during a CT imaging acquisition utilizing a polychromatic X-ray source and a photon counting detector, across a plurality of energy bins based on energies of the detected X-ray photons, apply a set of reconstruction bin weights to the plurality of energy bins, wherein the set of reconstruction bin weights are configured to enhance one or more K-edges, suppress one or more K-edges, or enhance one or more K-edges and suppress one or more K-edges, combine the weighted energy bins into a single set of data, and reconstruct the single set of data to generate K-edge volumetric image data that enhances at least one K-edge.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an imaging system configured for spectral computed tomography (CT) imaging and including a set of reconstruction bin weights, in accordance with an embodiment(s) herein.

FIG. 2 graphically illustrates K-edges for certain materials, such Iodine, Gadolinium, and Tantalum, which are utilized as radiocontrast agents in CT imaging, in plots of linear attenuation coefficient versus photon energy plots.

FIG. 3 graphically illustrates example energy bins for enhancing and/or suppressing a K-edge of Iodine for a scan including a single radiocontrast agent, in accordance with an embodiment(s) herein.

FIG. 4 graphically illustrates example energy bins for enhancing and/or suppressing a K-edge of Gadolinium for a scan including a single radiocontrast agent, in accordance with an embodiment(s) herein.

FIG. 5 graphically illustrates example energy bins for enhancing and/or suppressing a K-edge of Tantalum for a scan including a single radiocontrast agent, in accordance with an embodiment(s) herein.

FIG. 6 graphically illustrates an example set of energy bins for enhancing and/or suppressing one or more K-edges for a scan including two radiocontrast agents, in accordance with an embodiment(s) herein.

FIG. 7 graphically illustrates another example set of energy bins for enhancing and/or suppressing one or more K-edges for a scan including two radiocontrast agents, in accordance with an embodiment(s) herein.

FIG. 8 graphically illustrates yet another example set of energy bins for enhancing and/or suppressing one or more three K-edges for a scan including at least three radiocontrast agents, in accordance with an embodiment(s) herein.

FIG. 9 depicts an image slice of a phantom from a reconstruction that employs nominal bin weights for a weighted sum reconstruction.

FIG. 10 depicts the image slice of the phantom from a reconstruction that employs energy bin weights to enhance a set of K-edge materials, in accordance with an embodiment(s) herein

FIG. 11 depicts the image slice of the phantom from a reconstruction that employs a different set of energy bin weights to enhance a set of K-edge materials, in accordance with an embodiment(s) herein

FIG. 12 illustrates a non-limiting example of a flow chart for a computer-implemented method for generating K-edge enhanced volumetric image data based on reconstructing a weighted summation of a plurality of energy bins, in accordance with an embodiment(s) herein.

### DETAILED DESCRIPTION

Embodiments of the present disclosure will now be described, by way of example, with reference to the figures, in which a system, a method and/or instructions on a computer readable medium for reconstructing energy-binned spectral projection data, which is acquired using a polychromatic X-ray source and a photon counting detector, includes using a set of predetermined energy bin weights for the counts in the energy bins to generate K-edge volumetric image data, which enhances and/or suppresses one or more radiocontrast agents of interest in the volumetric image data based on the K-edges of the one or more radiocontrast agents of interest. The set of predetermined energy bin weights can be empirically and/or otherwise determined, and/or static and/or dynamically determined.

As described in greater detail below, in one instance this includes setting a K-edge energy threshold boundary that is shared by a pair of neighboring narrow energy windows of a pair of neighboring energy bins at a K-edge of a radiocontrast agent of interest, weighting the counts in the pair of bins differently, as well as different from the counts in the other bins, to enhance the counts, combining the weighted counts into a single set of projection data (e.g., a sinogram, etc.), and reconstructing the single set of projection data to generate K-edge enhanced volumetric image data that enhances the radiocontrast agent. Where multiple radiocontrast agents are employed for a scan, a radiocontrast agent can be enhanced either by directly enhancing the counts associated with the radiocontrast agent and/or suppressing the counts for another radiocontrast agent utilized for the scan.

As discussed above, with one existing K-edge image subtraction approach, an image is reconstructed with the counts from an energy bin with an upper energy threshold at the K-edge energy and another image is reconstructed with the projection data from a neighboring energy bin with a lower energy threshold at the K-edge energy. The two reconstructed images are then subtracted to create a K-edge subtraction image. However, such an approach requires at least two reconstructions, one for each of the two images, and, unfortunately, the additional reconstruction consumes additional processing resources and/or delays generation of final volumetric image data, and/or the at least two reconstructions do not utilize the counts from all of the bins, which increases noise and/or artifact, which degrades the image quality, e.g., to the point of impractical or unworkable image quality.

With the approach described herein, only a single reconstruction is performed to generate the K-edge enhanced volumetric image data, mitigating any need for additional processing resources (e.g., memory, processing cycles, etc.) and/or delays in the generation of the final K-edge enhanced volumetric image data associated with approaches requiring more than one reconstruction. Furthermore, the counts of more than just the two bins about the K-edge threshold (e.g., even all of the bins) are combined into a single set of projection data and reconstructed, providing image quality for diagnostic qualitative imaging. Furthermore, in one instance, the approach allows for reducing an amount of radiocontrast agent utilized for a scan, which may reduce or eliminate risks for radiocontrast agent contraindicated patients.

Additionally, or alternatively, some spectral CT imaging systems are configured to generate spectral volumetric image data (including K-edge material volumetric image data) by combining one or more material decomposition algorithms (which leverage the different attenuation properties of materials at varying energy levels) with the reconstruction. Such material decomposition algorithms can be computationally and/or time-intensive. The approach described herein generates K-edge enhanced volumetric image data without a material decomposition. As such, the approach described herein simplifies the image reconstruction process relative to a configuration that utilizes material decomposition and mitigates a need for the additional processing resources and/or time associated with imaging systems employing a material decomposition.

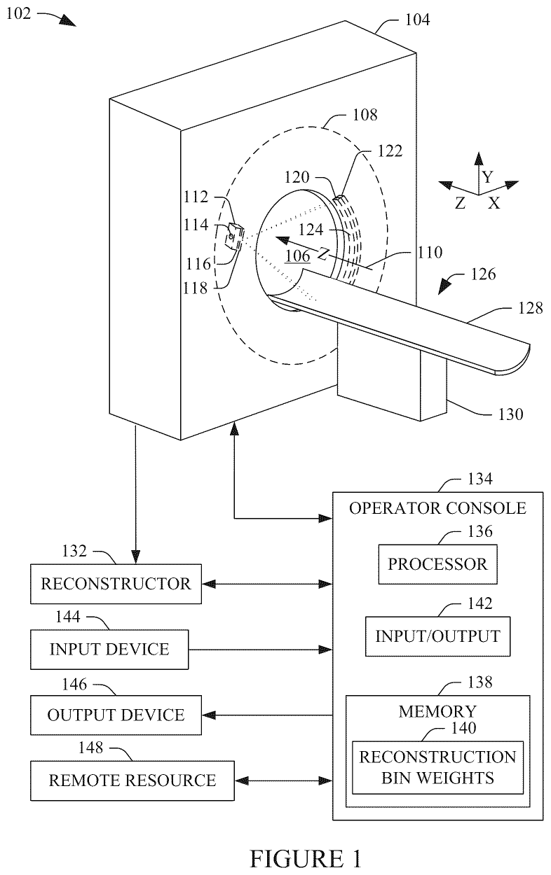

Initially referring to FIG. 1, a non-limiting example of an imaging system 102 configured at least for spectral computed tomography (CT) imaging is schematically illustrated. The imaging system 102 includes a gantry 104 with a bore 106 and a rotating frame 108. The rotating frame 108 is rotatably supported by the gantry 104, e.g., via a bearing (e.g., a slip ring, etc.) or the like, and is configured to rotate around the bore 106 about a rotational or z-axis 110, which extends through a center of rotation (e.g., a center of the bore 106, i.e., an isocenter). In some instances, the gantry 104 can be configured to tilt through the z-axis 110. A rotating frame controller is configured to control rotation (and tilt, if available) of the rotating frame 108, including no rotation.

The rotating frame 108 includes components utilized in the generation, emission and detection of X-rays. For instance, an X-ray source assembly 112 is supported by the rotating frame 108 and rotates in coordination with the rotating frame 108. The X-ray source assembly 112 includes an X-ray source 114 such as an X-ray tube and/or other source that generates and/or emits X-ray radiation. The illustrated X-ray source 114 includes at least an X-ray tube that is configured to emit polychromatic broadband X-ray radiation having an energy at least in the X-ray diagnostic range such as twenty (20) kiloelectronvolts (keV) to one-hundred and fifty (150) keV), more, or less. The X-ray assembly 112 further includes or is coupled to a filter 116, such as a filter that characterizes a radiation dose profile. The X-ray assembly 112 further includes or is coupled to a collimator 118 that shapes the X-ray radiation to form a generally fan, wedge, cone, etc. shaped beam that traverses the bore 106. An X-ray controller is configured to control components of the X-ray assembly 112, including the X-ray source 114 (e.g., such as kVp, mAs, etc.), the collimator 118 (e.g., a width of the X-ray beam), etc.

The components carried by the rotating frame 108 further include a detector array 120 and a Data Acquisition System (DAS) 122. The detector array 120 and the DAS 122 are supported by the rotating frame 108, opposite the X-ray source 114 along an arc, across the bore 106, and rotate in coordination with the rotating frame 108, including the X-ray source 114. The detector array 120 includes a one-dimensional (1-D) and/or a two-dimensional (2-D) array of rows of X-ray radiation sensitive detector elements 124. The X-ray radiation sensitive detector elements 124 are in electrical communication with the DAS 122. The illustrated X-ray radiation sensitive detector elements 124 include a direct conversion photon counting detector such as a silicon (Si), a Cadmium Telluride (CdTe), a Cadmium Zinc Telluride (CZT), etc. based detector. Such materials directly convert X-ray photons to electrical signals indicative of an energy of the X-ray photon. The individual detected X-ray photons are grouped into a plurality of energy bins based on the signals. A DAS controller (not visible) controls the detector array 120, the X-ray radiation sensitive detector elements 124, the DAS 122, etc.

The imaging system 102 further includes a table 126. The table 126 includes a cradle 128 moveably coupled to a frame/base 130. In one instance, the cradle 128 is slidably coupled to the frame/base 130 via a bearing or the like, and a drive system (not visible) including a motor, a lead screw, and a nut (or other drive system) translates the cradle 128 along the frame/base 130 into and out of the bore 106 for horizontal motion, and the frame/base 130 includes a drive system (not visible) including a mechanism for vertical or diagonal motion. The cradle 128 is configured to support a subject in the bore 106 for loading, scanning, and/or unloading. A table controller (not visible) controls the drive system of the table 126.

For an axial (step and shoot) scan, the cradle 128 is positioned at a static position for each integration period and moves between integration periods. For a helical scan, the rotating frame 108 rotates in coordination with the cradle 128 moving along the Z-axis 110, and active X-ray radiation sensitive detector elements 124 of the detector array 120 detect X-ray radiation over consecutive arc segments (integration periods) with each revolution and generate respective signals. For each arc segment, the DAS 122 processes each signal and generates projection data.

A reconstructor 132 reconstructs the projection data and generates 3-D volumetric image data for a helical scan and/or an individual axial (2-D) slice for an axial step and shoot scan (which can be used in combination to generate volumetric image data). The 3-D volumetric image data and/or 2-D slices thereof, and/or the individual axial images can be visually presented, filmed, etc. The reconstructor 132 employs predetermined reconstruction bin weights, depending on the reconstruction. For example, with K-edge imaging, in one instance, the reconstruction bin weights are configured to enhance and/or suppress one or more K-edges of one or more radiocontrast agents, as described in greater detail below. In one instance, the reconstructor 132 additionally, or alternatively, is further configured to employ material decomposition to generate spectral volumetric image data. Examples of suitable reconstruction algorithms include filtered back projection (FBP), advanced statistical iterative reconstruction (ASIR), conjugate gradient (CG), maximum likelihood expectation maximization (MLEM), model-based iterative reconstruction (MBIR), and/or other reconstruction algorithms.

A computing system 134 serves as an operator console of the imaging system 102. The computing system 134 includes a computer, a workstation, server, etc. The computing system 134 includes a processor 136 such as a microprocessor (mP), a central processing unit (CPU), graphics processing unit (GPU), etc., and a computer readable medium 138 (“MEMORY”), which includes non-transitory medium and excludes transitory medium (signals, carrier waves, and the like). The computer readable medium/memory 138 at least includes reconstruction bin weights 140.

The reconstruction bin weights 140 are utilized by the reconstructor 132 to weight the counts in different energy bins before the counts are combined into a single set of projection data and reconstructed to generate K-edge enhanced volumetric image data. The reconstruction bin weights 140 can be determined empirically and/or otherwise (and can be static and/or dynamic) based on the different energy bins and the K-edges of one or more radiocontrast agents to enhance and/or suppress one or more radiocontrast agents in the reconstructed K-edge enhanced volumetric image data. In one instance, the reconstruction bin weights 140 are configured to directly enhance a particular radiocontrast agent, and, in another instance, the reconstruction bin weights 140 are configured to suppress a particular radiocontrast agent to indirectly enhance a different radiocontrast agent. This includes enhancing one or more radiocontrast agents and/or suppressing one or more radiocontrast agents for a scan involving one or more radiocontrast agents.

As discussed above, an existing K-edge image subtraction approach requires multiple reconstructions and reconstructs counts only from two energy bins. The former consumes additional processing resources and/or delays generation of final volumetric image data, and the latter increases noise and/or artifact in the final volumetric image data. The approach described herein includes a single reconstruction using weighted counts from multiple energy bins (e.g., more than two, all, etc.), mitigating any need for additional processing resources and/or delays in the generation of the final K-edge volumetric image data, and providing image quality (e.g., good noise statistics) for diagnostic qualitative imaging. The approach allows for reducing an amount of radiocontrast agent utilized for a scan, which may reduce or eliminate risks for radiocontrast agent contraindicated patients.

The computing system 134 further includes input/output (I/O) 142. An input device 144 includes a keyboard, mouse, touchscreen, microphone, etc. In one instance, a user employes the input device 144 to select a protocol that indicates a set of reconstruction bin weights of the reconstruction bin weights 140 for the scan. In another instance, a user employes the input device 144 to indicate a radiocontrast agent of interest, and a set of reconstruction bin weights of the reconstruction bin weights 140 for the scan are determined based on the radiocontrast agent of interest. The input device 144 and/or other device may be utilized to adjust the bins and/or the bin weights. An output device 146 includes a human readable device such as a display monitor or the like. The computing system 134, the input device 144 and/or the output device 146 are in electrical communication with the computing system 134 through the I/O 142 and/or otherwise.

A remote resource 148 includes one or more of a server, a workstation, a Radiology Information System (RIS), a Hospital Information System (HIS), an Electronic Medical Record (EMR), a Picture Archiving and Communication System (PACS) for storing information, a PACS further configured with image viewing and/or manipulating software, cloud resources with shared remote data storage and/or computing power including resources distributed over data centers, etc. The computing system 134 and the remote resource 148 are in communication via wired and/or wireless technologies. Such communication can be through Digital Imaging and Communications in Medicine (DICOM), Health Level Seven (HL7), etc. formats and protocols.

As discussed herein, in general, the absorption of X-ray photons by a material decreases with increasing X-ray photon energy. However, certain materials include a temporary and measurable increase in the absorption of X-rays with increasing X-ray photon energy at the K-edge energy of a material due to the ejection of an electron from the K-shell. The K-edge energy of such a material is unique to and characteristic of the material. The K-edge properties of certain materials render them suitable for use as CT radiocontrast agents. For example, the diagnostic energy range for CT is around 20 keV to 150 keV, and examples of CT radiocontrast agents include Iodine (I), Gadolinium (Gd), Tantalum (Ta), etc. I (Z=53) has a K-edge at approximately 33.2 keV, Gd (Z=64) has a K-edge at approximately 50.2 keV, and Ta (Z=73) has a K-edge at approximately 67.4 keV.

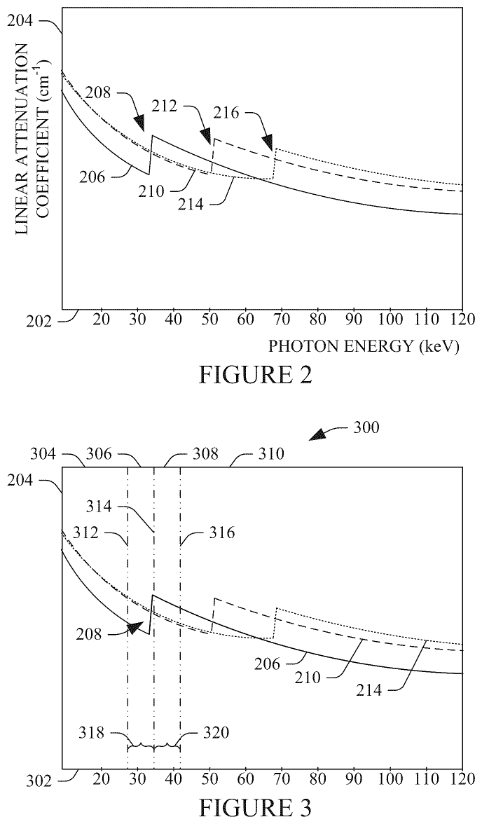

FIG. 2 graphically illustrates K-edges for I, Gd, and Ta via linear attenuation coefficient versus energy plots. A first axis 202 represents photon energy in units of keV. The energy range in this example (20 keV to 120 keV) covers a portion of the CT diagnostic energy range (20 keV to 150 keV). A second axis 204 represents the linear attenuation coefficient in units of inverse centimeters (cm⁻¹). A first plot 206 represents the linear attenuation coefficient of I as a function of X-ray photon energy. From the first plot 206, in general, the linear attenuation coefficient of I decreases with increasing X-ray photon energy. However, at the K-edge energy of I (~33.2 keV), there is a temporary and measurable increase 208 in the linear attenuation coefficient of I.

A second plot 210 represents the linear attenuation coefficient of Gd as a function of X-ray photon energy. From the second plot 210, in general, the linear attenuation coefficient of Gd decreases with increasing X-ray photon energy. However, at the K-edge energy of Gd (~50.3 keV), there is a temporary and measurable increase 212 in the linear attenuation coefficient of Gd. A third plot 214 represents the linear attenuation coefficient of Ta as a function of X-ray photon energy. From the third plot 214, in general, the linear attenuation coefficient of Ta decreases with increasing X-ray photon energy. However, at the K-edge energy of Ta (~67.4 keV), there is a temporary and measurable increase 216 in the linear attenuation coefficient of Ta.

Materials such as I, Gd and Ta have been utilized as CT radiocontrast agents for applications such as cardiac applications, including functional cardiac applications such as perfusion imaging which measures how well blood flows through the heart muscle and/or structural cardiac applications such as coronary angiography which provides an anatomical characterization of the coronary arteries. Although the K-edges of I, Gd and Ta are briefly discussed above and FIG. 2 graphically illustrates K-edges for I, Gd, and Ta, other materials include K-edges, including other materials suitable as a CT radiocontrast agent. For example, another material utilized as a CT radiocontrast agent is Barium (Ba, Z=56), which has a K-edge at approximately 37.4 keV. Other materials have K-edges outside of the CT diagnostic energy range.

In one instance, each photon counting detector of the detector elements 124 is configured with at least four energy bins, and the reconstruction bin weights 140 include a set of reconstruction bin weights for enhancing and/or suppressing one or more particular K-edges. With four energy bins, a single K-edge can be enhanced and/or suppressed. With each additional two energy bins, another K-edge can be enhanced and/or suppressed. For example, with eight energy bins, up to three different K-edges can be enhanced and/or suppressed. FIGS. 3, 4 and 5 graphically illustrate example energy bins for enhancing and/or suppressing a single K-edge for a scan including a single radiocontrast agent.

Initially referring to FIG. 3, the plots 206, 210 and 214 and K-edges 208, 212 and 216 described in connection with FIG. 2 are shown in connection with a set of energy bins 300 for explanatory purposes. Again, the first plot 206 represents the linear attenuation coefficient of I as a function of X-ray photon energy, the second plot 210 represents the linear attenuation coefficient of Gd as a function of X-ray photon energy, and the third plot 214 represents the linear attenuation coefficient of Ta as a function of X-ray photon energy. A first axis 302 represents photon energy in units of keV.

In this example, there are at least four (4) energy bins, including a first bin 304, a second bin 306, a third bin 308, and a fourth bin 310. In this example, the first bin 304 and/or the fourth bin 310 are each single bins and/or a combination of bins. In another example, at least one of the first bin 304 and/or the fourth bin 310 includes multiple individual and different bins. In this example, there are at least three (3) energy thresholds, including a first energy threshold 312, a second energy threshold 314, and a third energy threshold 316. In general, for N bins (where N is a positive integer), there are N-1 energy thresholds.

The second energy threshold 314 is set in accordance with the K-edge of I. In this example, the second energy threshold 314 is set just after the K-edge of I. In another example, the second energy threshold 314 is set just before the K-edge of I. In yet another example, the second energy threshold 314 is set approximately on the K-edge of I. Where the bins 304 and 310 each include a single bin, the first bin 304 includes an upper energy threshold, which is the first energy threshold 312, and the fourth bin 310 includes a lower energy threshold, which is the third energy threshold 316.

The second bin 306 includes an energy window 318. The energy window 318 of the second bin 306 includes a lower energy threshold, which is the first energy threshold 312, and an upper energy threshold, which is the second energy threshold 314. The third bin 308 includes an energy window 320. The energy window 320 of the third bin 308 includes a lower energy threshold, which is the second energy threshold 314, and an upper energy threshold, which is the third energy threshold 316. In this example, the second bin 306 and the third bin 308 (i.e. the energy window 318 and the energy window 320) share the second energy threshold 314. The widths of the bins 306 and 308 (i.e., the energy windows 318 and 320) can be the same or different. Examples of suitable bin width sizes include a bin width size in a range of 5 keV-20 keV, such as 7 keV, 17 keV, etc. In general, wide energy windows reduce the advantage provided by the increase at the K-edge energy.

In this example, X-ray photons having an energy less than the first energy threshold 312 are registered with the first bin 304, and X-ray photons having an energy greater than the third energy threshold 316 are registered with the fourth bin 310. X-ray photons having an energy within the energy window 318 are registered with the second bin 306, and X-ray photons having an energy within the energy window 320 are registered with the third bin 308. X-ray photons having an energy at the second energy threshold 314 can be split between the bins 306 and 308, registered with one of the bins 306 and 308, and/or otherwise binned.

With this example, in one instance, to enhance the K-edge of I, the bins 304 and 310 are weighted with a nominal weight (e.g., 1.0), the second bin 306 is weighted with a negative weight L, and the third bin 308 is weighted with a positive weight M that is greater than the nominal weight. In general, the weight for the bin after the K-edge threshold will be larger than the weight for the bin before the K-edge threshold to enhance the K-edge. In this example, the weights L and M have an absolute value that is greater than one (L and M>1.0) to amplify the K-edge relative to the other bins. Examples of weights of the weight L include −2.0, −2.5, −4.0, etc., and examples of weights of the weight M include 2.0, 2.5, 5.0, etc. The weights for L and M can have the same or different absolute value, but M is larger than L (M>L) to enhance the K-edge.

The reconstructor 132 respectively applies the weights to the counts in each of the bins 304, 306, 308 and 310, combines with the weighted counts into a single set of projection data (e.g., a sinogram, etc.), and reconstructs the weighted sum of the counts to generate K-edge enhanced (I in this example) volumetric image data. As described herein, this approach mitigates consuming additional processing resources (e.g., memory, processing cycles, etc.) and/or delays in the generation of the final volumetric image data associated with approaches requiring more than one reconstruction, and/or reduces noise and/or artifact relative to a configuration in which counts from only a single bin are utilized.

For non-radiocontrast agent enhanced scans, a same weight value (e.g., 1.0) can be applied to all of the bins. For example, where the operator selects a radiocontrast agent enhanced scan, a set of reconstruction bin weights configured to enhance the K-edge is provided to the reconstructor 132, and where the operator selects a non-radiocontrast agent enhanced scan, a different set of reconstruction bin weights that do not enhance the K-edge is provided to the reconstructor 132. The particular set of reconstruction bin weights for the radiocontrast agent enhanced scan may depend on the radiocontrast agent and/or may be independent of the radiocontrast agent. In some instances, there are more than one set of bins and/or weights for radiocontrast agent enhanced scans, and/or more than one set of bins and/or weights for non-radiocontrast agent enhanced scans.

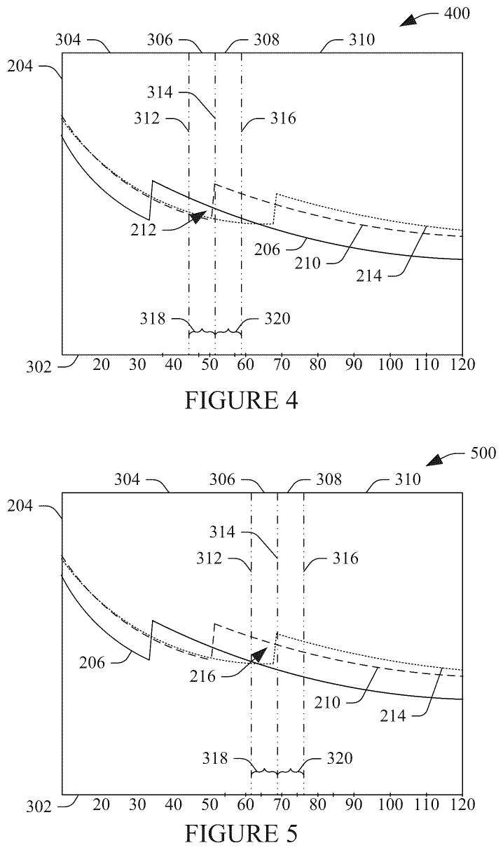

FIG. 4 graphically illustrates example energy bins for enhancing and/or suppressing a single K-edge for a scan including a single radiocontrast agent. Again, the plots 206, 210 and 214 described in connection with FIG. 2 are shown in connection with a set of energy bins 400 for explanatory purposes. FIG. 4 is substantially similar to the example described in connection with FIG. 3 except that with the bins 304, 306, 308 and 310, the thresholds 312, 314 and 316, and the energy windows 318 and 320 are reconfigured from the K-edge of I to the K-edge of Gd to enhance and/or suppress Gd. In FIGS. 3 and 4, the bins 306 and 308 can be a same or different size. Furthermore, additional bins for energy-discrimination can be included.

FIG. 5 graphically illustrates example energy bins for enhancing and/or suppressing a single K-edge for a scan including a single radiocontrast agent. Again, the plots 206, 210 and 214 described in connection with FIG. 2 are shown in connection with a set of energy bins 500 for explanatory purposes. FIG. 5 is substantially similar to the example described in connection with FIG. 3 except that with the bins 304, 306, 308 and 310, the thresholds 312, 314 and 316, and the energy windows 318 and 320 are reconfigured from the K-edge of I to the K-edge of Ta to enhance and/or suppress Ta. In FIGS. 3, 4 and 5, the bins 306 and 308 can be a same or different size. Furthermore, additional bins for energy-discrimination can be included.

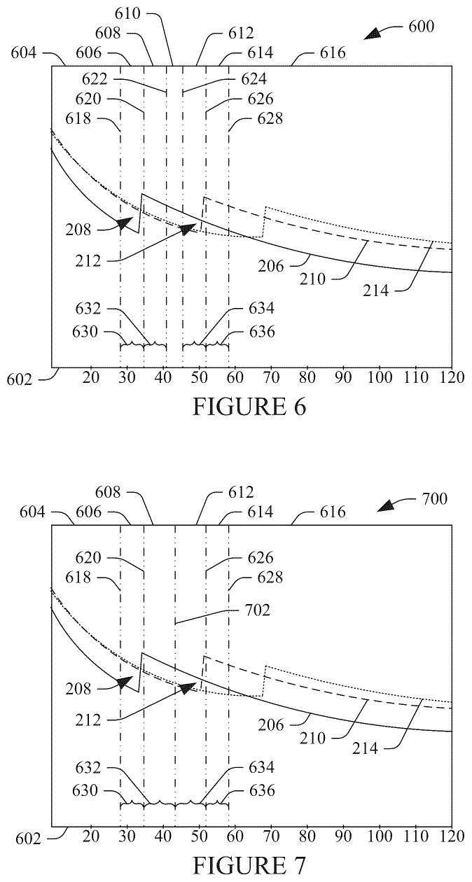

FIGS. 6 and 7 graphically illustrate example energy bins for enhancing and/or suppressing one or more K-edges for a scan including two radiocontrast agents. Again, the plots 206, 210 and 214 described in connection with FIG. 2 are shown in connection respectively with a set of energy bins 600 and 700 for explanatory purposes, where the first plot 206 represents the linear attenuation coefficient of I as a function of X-ray photon energy, the second plot 210 represents the linear attenuation coefficient of Gd as a function of X-ray photon energy, and the third plot 214 represents the linear attenuation coefficient of Ta as a function of X-ray photon energy.

Initially referring to FIG. 6, a first axis 602 represents photon energy in units of keV. There are at least seven (7) energy bins, including a first bin 604, a second bin 606, a third bin 608, a fourth bin 610, a fifth bin 612, a sixth bin 614, and a seventh bin 616. In one example, the first bin 604 and/or the seventh bin 616 are each single bins and/or a combination of multiple bins. In another example, at least one of the first bin 604 and/or the seventh bin 616 includes multiple different bins. In this example, there are six (6) energy thresholds, including a first energy threshold 618, a second energy threshold 620, a third energy threshold 622, a fourth energy threshold 624, a fifth energy threshold 626, and a sixth energy threshold 628.

The second energy threshold 620 is set in accordance with the K-edge of I, as described in connection with K-edge energy threshold described in connection with FIG. 3, and the fifth energy threshold 626 is set in accordance with the K-edge of Gd, as described in connection with K-edge energy threshold described in connection with FIG. 4. Where the bins 604 and 616 each include a single bin, the first bin 604 includes an upper energy threshold, which is the first energy threshold 618, and the seventh bin 616 includes a lower energy threshold, which is the sixth energy threshold 628. The fifth bin 610 includes a lower energy threshold, which is the third energy threshold 622, and an upper energy threshold, which is the fourth energy threshold 624.

The second bin 606 includes an energy window 630. The energy window 630 of the second bin 606 includes a lower energy threshold, which is the first energy threshold 618, and an upper energy threshold, which is the third energy threshold 622. The third bin 608 includes an energy window 632. The energy window 632 of the second bin 606 includes a lower energy threshold, which is the second energy threshold 620, and an upper energy threshold, which is the third energy threshold 622. In this example, the second bin 606 and the third bin 608 share the second energy threshold 620, which is at the K-edge energy of I.

The fifth bin 612 includes an energy window 634. The energy window 634 of the fifth bin 612 includes a lower energy threshold, which is the fourth energy threshold 624, and an upper energy threshold, which is the fifth energy threshold 626. The sixth bin 614 includes an energy window 636. The energy window 636 of the sixth bin 614 includes a lower energy threshold, which is the fifth energy threshold 626, and an upper energy threshold, which is the sixth threshold 628. In this example, the fifth bin 612 and the sixth bin 614 share the fifth energy threshold 626, which is at the K-edge energy of Gd.

In this example, X-ray photons having an energy less than the first energy threshold 618 are registered with the first bin 604, and X-ray photons having an energy greater than the sixth energy threshold 628 are registered with the seventh bin 616. X-ray photons having an energy within the energy window 630 are registered with the second bin 606, and X-ray photons having an energy within the energy window 634 are registered with the third bin 608. X-ray photons having an energy within the energy thresholds 622 and 624 are registered with the fourth bin 610. X-ray photons having an energy within the energy window 634 are registered with the fifth bin 612, and X-ray photons having an energy within the energy window 634 are registered with the sixth bin 614.

With this example, in one instance, to enhance the K-edge of I, at least the bins 606 and 608 are weighted as described in connection with the description of the FIG. 3. Additionally, or alternatively, at least the bins 612 and 614 are weighted to suppress the K-edge of Gd to enhance the K-edge of I. In another example, in one instance, to enhance the K-edge of Gd, at least the bins 612 and 614 are weighted as described in connection with the description of the FIG. 4. Additionally, or alternatively, at least the bins 606 and 608 are weighted to suppress the K-edge of I to enhance the K-edge of Gd. In another instance, at least the bins 606, 608, 612 and 616 are weighted to enhance and/or suppress both the K-edge of I and the K-edge of Gd.

As described in connection with FIG. 3, the weighted counts are combined into a single set of project data, and the weighted sum is reconstructed to generate K-edge enhanced volumetric image data. Again, this approach mitigates consuming additional processing resources (e.g., memory, processing cycles, etc.) and/or delays in the generation of the final volumetric image data associated with approaches requiring more than one reconstruction, and/or reduces noise and/or artifact relative to a configuration in which counts from only a single bin are utilized.

For non-radiocontrast agent enhanced scans, a same weight value (e.g., 1.0) can be applied to all of the bins. The particular set of reconstruction bin weights for the radiocontrast agent enhanced scan may depend on the radiocontrast agent and/or may be independent of the radiocontrast agent. In some instances, there are more than one set of bins and/or weights for radiocontrast agent enhanced scans, and/or more than one set of bins and/or weights for non-radiocontrast agent enhanced scans.

Briefly turning to FIG. 7, another set of energy bins 700 are graphically illustrated in connection with the plots 206, 210 and 214. In FIG. 6, there was a bin (the fourth bin 610) between the pair of bins 606/608 for the K-edge of I and the pair of bins 612/614 for the K-edge of Gd. FIG. 7 is substantially similar to the example described in connection with FIG. 6 except that the third and fourth energy thresholds 622 and 624 are omitted, and a single energy threshold 702 is shared by the bins 608 and 612. The widths of energy windows 630, 632, 634 and 636 can be similar and/or different.

With the examples described in connection with FIGS. 6 and 7, the K-edge thresholds are set in accordance with the K-edges of I and Gd. In another instance, the K-edge thresholds are set in accordance with the K-edges of I and Ta. In yet another instance, the K-edge thresholds are set in accordance with the K-edges of Ta and Gd. For this, similar to the example described in connection with FIGS. 4 and 5, a set of the bins, the thresholds, and the energy windows are accordingly set-essentially reconfigured from the K-edge of Gd to the K-edge of Ta, from the K-edge of I to the K-edge of Ta, and/or otherwise, and/or additional bins can be added.

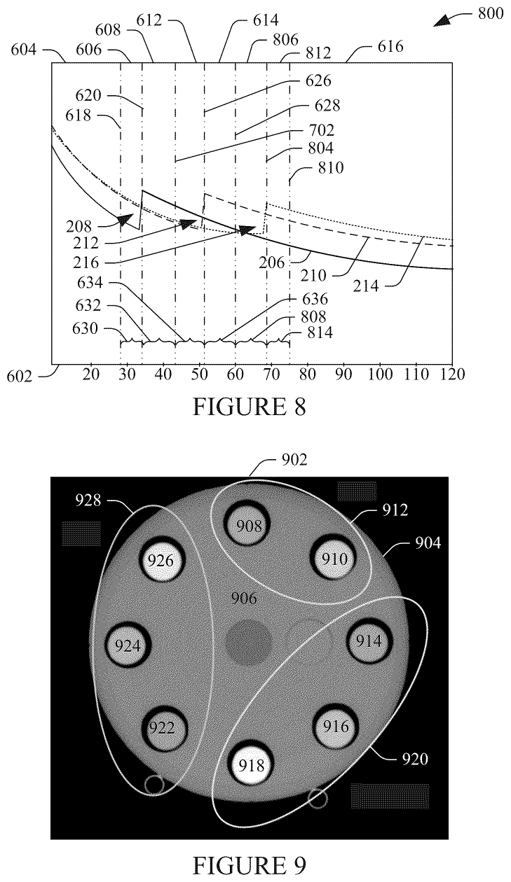

FIG. 8 is substantially similar to FIG. 7 except another pair of bins are included for another K-edge. Again, the plots 206, 210 and 214 described in connection with FIG. 2 are shown in connection with a set of energy bins 800 for explanatory purposes, where the first plot 206 represents the linear attenuation coefficient of I as a function of X-ray photon energy, the second plot 210 represents the linear attenuation coefficient of Gd as a function of X-ray photon energy, and the third plot 214 represents the linear attenuation coefficient of Ta as a function of X-ray photon energy.

In FIG. 8, an energy threshold 804 is set in accordance with the K-edge of Ta. A bin 806 includes an energy window 808, which includes a lower energy threshold, which is the energy threshold 628, and an upper energy threshold, which is the energy threshold 804. An energy threshold 810 defines a bin 812, which includes an energy window 814. The window 814 includes a lower energy threshold, which is the energy threshold 804, and an upper energy threshold, which is the energy threshold 810. Depending on the reconstruction bin weights, one or more of the K-edges of I, Gd and/or Ta can be enhanced and/or suppressed as described herein.

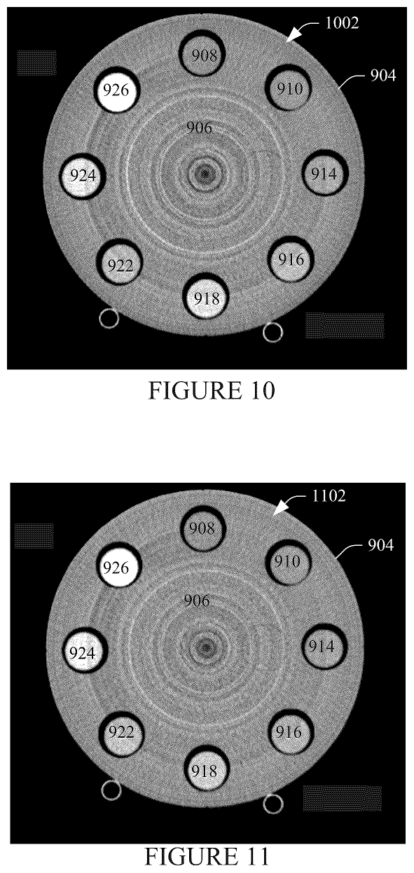

FIGS. 9, 10 and 11 depict an example of the approach described herein. FIG. 9 depicts an image slice of a phantom from a reconstruction that employs nominal reconstruction bin weights. FIGS. 10 and 11 depict the image slice from a reconstruction that employs reconstruction bin weights to enhance and/or suppress a material. FIG. 10 depicts the image slice with one set of reconstruction bin weights that enhances and/or suppress a material, and FIG. 11 depicts the image slice with a different set of reconstruction bin weights that enhances and/or suppress a material. In general, the phantom is a cylindrical CT phantom that includes a plurality of radiocontrast rods, each rod consisting of either I, Gd or Ta, and each rod including a specific and known concentration of I, Gd or Ta.

In initially referring to FIG. 9, an image slice 902 of a cylindrically shaped phantom 904 includes a first region 906 with a reference material with X-ray absorption characteristics similar to X-ray absorption characteristics of soft tissue. The cylindrically shaped phantom further includes a second region 908 that includes a first concentration of Gd. The cylindrically shaped phantom further includes a third region 910 that includes second concentration of Gd, where the second concentration of Gd is greater than the first concentration of Gd. The second region 908 and the third region 910 are collectively referred to herein as Gd regions 912.

The phantom further includes a fourth region 914 that includes a first concentration of I, a fifth region 916 that includes a second concentration of I, and a sixth region 918 that includes third concentration of I, where the third concentration is greater than the second concentration, which is greater than the first concentration. The fourth region 914, the fifth region 916 and the sixth region 918 are collectively referred to herein as I regions 920. The phantom further includes a seventh region 922 that includes a first concentration of Ta, an eighth region 924 that includes a second concentration of Ta, and a ninth region 926 that includes third concentration of Ta, where the third concentration is greater than the second concentration, which is greater than the first concentration. The seventh region 922, the eighth region 924 and the ninth region 926 are collectively referred to herein as Ta regions 928.

In this example, the concentrations of the radiocontrast agents in the second region 908, the fourth region 914 and the seventh region 922 are approximately the same, the concentrations of the radiocontrast agents in the third region 910, the fifth region 916 and the eighth region 924 are approximately the same, and the concentrations of the radiocontrast agents in the sixth region 918 and the ninth region 926 are approximately the same. The nominal reconstruction bin weights utilized for all of the bins is 1.0. In other instance, different nominal reconstruction bin weights are utilized.

With such weights, the Ta, I and Gd contrasts respectively in the seventh region 922, the fourth region 914 and the second region 908 are visually similar, and an observer would not be able to readily distinguish which region included which material. That is, without knowing a priori the material in each rod, the observer would not be able to readily distinguish between the Ta, I and Gd regions. For a scan employing two radiocontrast agents such as I for functional scanning (e.g., perfusion, etc.) and Gd for structural scanning (e.g., coronary arteries, etc.), the functional and structural information in the reconstructed image data would not be visually distinguishable or separatable.

Likewise, the Ta, I and Gd contrasts respectively in the eighth region 924, the fifth region 916 and the third region 910 are visually similar, and an observer would not be able to readily distinguish which region included which material. That is, without knowing a priori the material in each rod, the observer would not be able to readily distinguish between the Ta, I and Gd regions. Again, for a scan employing two radiocontrast agents such as I for functional scanning (e.g., perfusion, etc.) and Gd for structural scanning (e.g., coronary arteries, etc.), the functional and structural information in the reconstructed image data would not be visually distinguishable or separatable.

Likewise, the Ta, I and Gd contrasts respectively in the ninth region 926 and the sixth region 918 are visually similar, and an observer would not be able to readily distinguish which region included which material. That is, without knowing a priori the material in each rod, the observer would not be able to readily distinguish between the Ta and I regions. Again, for a scan employing two radiocontrast agents such as I for functional scanning (e.g., perfusion, etc.) and Gd for structural scanning (e.g., coronary arteries, etc.), the functional and structural information in the reconstructed image data would not be visually distinguishable or separatable.

Next at FIG. 10, the set of reconstruction bin weights utilized in the reconstruction included a predetermined set of reconstruction bin weights that enhance Ta. In the illustrated example, the reconstruction bin weights were-2.0 for counts in the bin directly preceding the K-edge energy of Ta (67.4keV) and 2.0 for the counts in the bin directly following the K-edge. The reconstruction bin weights for the remaining bins are 1.0. In the context of FIG. 3 (discussed in detail above), the weight L for the second bin 306—2.0, the weight M for the third bin 308 is 2.0, and the weights for the bins 304 and 310 are 1.0. In FIG. 3, the upper energy threshold 314 of the energy window 318 for the bin 306 is the same as the lower energy threshold 314 of the energy window 320 for the bin 308, which is the threshold at the K-edge energy of Ta.

With such weights, the Ta radiocontrast in the seventh region 922 is increased, the I radiocontrast in the fourth region 914 is decreased, and Gd radiocontrast in the second region 908 is decreased (to about the reference material in this example), which, in combination visually enhances the Ta in seventh region 922 relative to the I in fourth region 914 and the Gd in second region 908. Similarly, the Ta radiocontrast in the eighth region 924 is increased, the I radiocontrast in the fifth region 916 is decreased, and the Gd radiocontrast in the third region 910 is decreased, which, in combination visually enhances the Ta in eighth region 924 relative to the I in the fifth region 916 and the Gd in third region 910, and the Ta radiocontrast in the ninth region 926 is increased and the I radiocontrast in the sixth region 918 is decreased, which, in combination visually enhances the Ta in the ninth region 926 relative to the I in the sixth region 918.

Next at FIG. 11, a set of bin weights utilized in the reconstruction include a predetermined set of bin weights that enhance Ta that are different than the set of bin weights discussed in connection with FIG. 10. In FIG. 10, the amplitude or absolute value of the bin weights around the K-edge of interest (the K-edge for Ta) are the same. In this example, the bin weights are −2.0 for counts in the bin directly preceding the K-edge, 5.0 for the counts in the bin directly following the K-edge, and 1.0 for the remaining bins. In the context of FIG. 3 (discussed in detail above), the weight L for the second bin 306—2.0, the weight M for the third bin 308 is 5.0, and the weights for the bins 304 and 310 are 1.0.

Similar to the weights discussed in connection with FIG. 10, the weights in FIG. 11 for corresponding regions increase the Ta radiocontrast and decrease the I radiocontrast and the Gd radiocontrast. However, in this particular example, the weights in FIG. 11 also reduce noise and image artifact 1102 (i.e., the radial bands-the concentric circles or rings radiating from a central point) relative to noise and image artifact 1002 (i.e., again, the radial bands) in FIG. 10. It is to be appreciated that the weights described in connection with FIGS. 9, 10 and 11 are for explanatory purposes and are not limiting, and in other instances different weights can be utilized, including different bin weights for one or more of the bins other than the pair of bins about the K-edge.

In one instance, this includes optimizing the energy bin weights to maximize contrast and/or minimize artifacts. This is achieved, in one instance, by varying the bin weights and measuring a contrast-to-noise ratio (CNR) of a K-edge contrast material and image quality metrics, e.g., pixel value uniformity across the image, ring detection metrics, etc., and utilizing the measured CNR and image quality metrics to determine energy bin weights that maximize contrast and/or minimize artifacts. Additionally, or alternatively, a mathematical model with an objective function configured to maximize contrast and/or minimize artifacts is utilized. Known and/or other approaches that optimize bin weights are also contemplated herein. A similar approach can be extended for multiple K-edge materials scenarios, where a CNR from one K-edge material is maximized while a CNR from another K-edge material is minimized, e.g., close to background material.

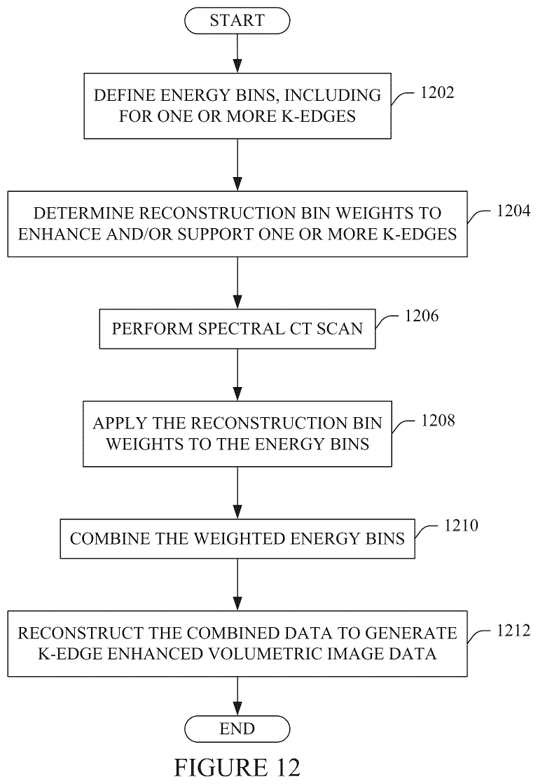

Moving to FIG. 12, a non-limiting example of a flow chart for a computer-implemented method for generating motion compensated 3-D volumetric image data is illustrated. It is to be appreciated that the ordering of the acts in the method is not limiting. As such, other orderings are contemplated herein. In addition, one or more acts may be omitted, and/or one or more additional acts may be included.

At 1202, a set of energy bins are defined for a photon counting detector, as described herein and/or otherwise. This includes defining at least one pair of bins in accordance with at least one K-edge, e.g., by setting an energy threshold at about the K-edge energy and thresholds to define an energy window about the K-edge energy, as described herein and/or otherwise. At 1204, a set of reconstruction bin weights for the set of energy bins are determined to enhance and/or suppress one or more K-edges of one or more radiocontrast agents, as described herein and/or otherwise.

At 1206, a spectral CT scan is performed using a polychromatic X-ray source and a photon counting detector where the subject being scanned is administered to one or more radiocontrast agents, as described herein and/or otherwise. At 1208, the set of reconstruction bin weights are applied to the set of energy bins, as described herein and/or otherwise. At 1210, the weighted set of energy bins are combined into a single set of projection data, as described herein and/or otherwise. At 1212, the single set of projection data is reconstructed to generate K-edge enhanced volumetric imaged data, as described herein and/or otherwise.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As utilized herein, the term controller includes computer-based devices with a microprocessor and/or the like and/or memory that is programmed to perform specific tasks. For example, the rotating frame controller is configured to control rotation of the rotating frame 108, the X-ray controller is configured to control X-ray emission, the DAS controller is configured to control the DAS 122, the table controller is configured to control the drive system of the table 126, etc.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A computed tomography imaging system configured for at least spectral imaging, comprising:
a polychromatic X-ray source;
a photon counting detector configured to detect X-ray photons traversing a subject and one or more radiocontrast agents administered to the subject and bin the detected X-ray photons across a plurality of energy bins based on energies of the detected X-ray photons,
wherein the one or more radiocontrast agents include one or more K-edges in the diagnostic imaging energy range;
memory including reconstruction bin weights configured to enhance one or more K-edges, suppress one or more K-edges, or enhance one or more K-edges and suppress one or more K-edges; and
a reconstructor configured to apply the reconstruction bin weights to the plurality of energy bins, combine the weighted energy bins into a single set of data, and reconstruct the single set of data to generate K-edge enhanced volumetric image data that enhances at least one K-edge.

2. The computed tomography imaging system of claim 1, wherein the plurality of energy bins include a first bin on a lower energy side of a first K-edge energy threshold for a first K-edge of interest, a second bin on an opposing higher energy side of the first K-edge energy threshold, and at least two other bins, wherein the reconstruction bin weights include a first weight for the first bin, a second weight for the second bin, and at least two other weights for the at least two other bins, and wherein a combination of the weighting of the first bin with the first weight and the weighting of the second bin with the second weight, relative to the weighting of the at least two other bins, enhances the first K-edge in the K-edge volumetric image data.

3. The computed tomography imaging system of claim 2, wherein the first weight down weights the first bin and the second weight upweights the second bin, relative to the at least two other weights and the at least two other bins.

4. The computed tomography imaging system of claim 2, wherein one of the first weight and the second weight is positive and the other of the first weight and the second weight is negative.

5. The computed tomography imaging system of claim 2, wherein an absolute value of the first weight and an absolute value of the second weight are the same.

6. The computed tomography imaging system of claim 2, wherein an absolute value of the first weight and an absolute value of the second weight are different.

7. The computed tomography imaging system of claim 2, wherein the at least two other weights have values of one.

8. The computed tomography imaging system of claim 2, wherein at least one of the at least two other weights have a value not equal to one.

9. The computed tomography imaging system of claim 2, wherein the plurality of energy bins further includes a third bin on a lower energy side of a second K-edge energy threshold for a second K-edge of interest and a fourth bin on an opposing higher energy side of the second K-edge energy threshold, wherein the reconstruction bin weights further include a third weight for the third bin and a fourth weight for the fourth bin, and wherein a combination of the weighting of the first bin with the first weight, the weighting of the second bin with the second weight, the weighting of the third bin with the third weight, the weighting of the fourth bin with the fourth weight, relative to the weighting of the at least two other bins, enhances the first K-edge in the K-edge volumetric image data.

10. The computed tomography imaging system of claim 9, wherein the third weight upweights the third bin and the fourth weight down weights the fourth bin to suppress the second K-edge to enhance the first K-edge in the K-edge volumetric image data.

11. The computed tomography imaging system of claim 10, wherein the first weight down weights the first bin and the second weight upweights the second bin to enhance the first K-edge in the K-edge volumetric image data, and the third weight down weights the third bin and the fourth weight upweights the fourth bin to enhance the second K-edge in the K-edge volumetric image data.

12. A computer-implemented method, comprising:
binning X-ray photons, which are detected during a CT imaging acquisition utilizing a polychromatic X-ray source and a photon counting detector, across a plurality of energy bins based on energies of the detected X-ray photons;
applying a set of reconstruction bin weights to the plurality of energy bins, wherein the set of reconstruction bin weights are configured to enhance one or more K-edges, suppress one or more K-edges, or enhance one or more K-edges and suppress one or more K-edges;
combining the weighted energy bins into a single set of data; and
reconstructing the single set of data to generate K-edge volumetric image data that enhances at least one K-edge.

13. The computer-implemented method of claim 12, wherein the plurality of energy bins include a first bin on a lower energy side of a first K-edge energy threshold for a first K-edge of interest, a second bin on an opposing higher energy side of the first K-edge energy threshold, and at least two other bins, wherein the reconstruction bin weights include a first weight for the first bin, a second weight for the second bin, and at least two other weights for the at least two other bins, and wherein a combination of the weighting of the first bin with the first weight and the weighting of the second bin with the second weight, relative to the weighting of the at least two other bins, enhances the first K-edge in the K-edge volumetric image data.

14. The computer-implemented method of claim 13, wherein the plurality of energy bins further includes a third bin on a lower energy side of a second K-edge energy threshold for a second K-edge of interest and a fourth bin on an opposing higher energy side of the second K-edge energy threshold, wherein the reconstruction bin weights further include a third weight for the third bin and a fourth weight for the fourth bin, and wherein a combination of the weighting of the first bin with the first weight, the weighting of the second bin with the second weight, the weighting of the third bin with the third weight, the weighting of the fourth bin with the fourth weight, relative to the weighting of the at least two other bins, enhances the first K-edge in the K-edge volumetric image data.

15. The computer-implemented method of claim 14, wherein the third weight upweights the third bin and the fourth weight down weights the fourth bin to suppress the second K-edge to enhance the first K-edge in the K-edge volumetric image data.

16. The computer-implemented method of claim 15, wherein the first weight down weights the first bin and the second weight upweights the second bin to enhance the first K-edge in the K-edge volumetric image data, and the third weight down weights the third bin and the fourth weight upweights the fourth bin to enhance the second K-edge in the K-edge volumetric image data.

17. A computer readable medium encoded with computer executable instructions, which, when executed by a processor, causes the processor to:
bin X-ray photons, which are detected during a CT imaging acquisition utilizing a polychromatic X-ray source and a photon counting detector, across a plurality of energy bins based on energies of the detected X-ray photons;
apply a set of reconstruction bin weights to the plurality of energy bins, wherein the set of reconstruction bin weights are configured to enhance one or more K-edges, suppress one or more K-edges, or enhance one or more K-edges and suppress one or more K-edges;
combine the weighted energy bins into a single set of data; and
reconstruct the single set of data to generate K-edge volumetric image data that enhances at least one K-edge.

18. The computer readable medium of claim 17, wherein the plurality of energy bins include a first bin on a lower energy side of a first K-edge energy threshold for a first K-edge of interest, a second bin on an opposing higher energy side of the first K-edge energy threshold, and at least two other bins, wherein the reconstruction bin weights include a first weight for the first bin, a second weight for the second bin, and at least two other weights for the at least two other bins, and wherein a combination of the weighting of the first bin with the first weight and the weighting of the second bin with the second weight, relative to the weighting of the at least two other bins, enhances the first K-edge in the K-edge volumetric image data.

19. The computer readable medium of claim 18, wherein the plurality of energy bins further includes a third bin on a lower energy side of a second K-edge energy threshold for a second K-edge of interest and a fourth bin on an opposing higher energy side of the second K-edge energy threshold, wherein the reconstruction bin weights further include a third weight for the third bin and a fourth weight for the fourth bin, and wherein a combination of the weighting of the first bin with the first weight, the weighting of the second bin with the second weight, the weighting of the third bin with the third weight, the weighting of the fourth bin with the fourth weight, relative to the weighting of the at least two other bins, enhances the first K-edge in the K-edge volumetric image data.

20. The computer readable medium of claim 19, wherein the third weight upweights the third bin and the fourth weight down weights the fourth bin to suppress the second K-edge to enhance the first K-edge in the K-edge volumetric image data, wherein the first weight down weights the first bin and the second weight upweights the second bin to enhance the first K-edge in the K-edge volumetric image data, and the third weight down weights the third bin and the fourth weight upweights the fourth bin to enhance the second K-edge in the K-edge volumetric image data.
